(19) United States
(12) Patent Application Publication
(10) Pub. No.: US 2026/0315130 A1
(43) Pub. Date: Oct. 15, 2026

(54) ABLATION SYSTEM

(71) Applicant: SHANGHAI MAAGI MEDICAL TECHNOLOGY CO., LTD, Shanghai (CN)

(72) Inventors: Kangwei ZHANG, Shanghai (CN); Aili ZHANG, Shanghai (CN); Xingchen WAN, Shanghai (CN); Jiajun GONG, Shanghai (CN)

(21) Appl. No.: 19/109,337

(22) PCT Filed: Nov. 16, 2022
(86) PCT No.: PCT/CN2022/132350

§ 371 Date: Mar. 6, 2025

(30) Foreign Application Priority Data
Sep. 6, 2022 (CN) 202211083012.6

(51) Int. Cl. A61B 18/02; A61B 18/00

(52) U.S. Cl. CPC A61B 18/02; A61B 2018/00172; A61B 2018/00577; A61B 2018/00642; A61B 2018/00714; A61B 2018/00744; A61B 2018/00791; A61B 2018/0262; A61B 2018/0293

(57) ABSTRACT

An ablation system includes a medium storage device used for storing a liquid medium; a plurality of probes each provided with an air inlet channel and an air return channel; a liquid inlet module provided with a gas-liquid separation device comprising a gas pipeline and a plurality of liquid pipelines, wherein each liquid pipeline can be communicated with or isolated from the air inlet channel of each probe; a heat exchange module provided with a heat exchanger, wherein the heat exchanger is communicated with the air return channel to convert the liquid medium delivered in the air return channel into a gas medium; and a control module electrically connected to each probe and the heat exchange module respectively.

The present application claims priority to Chinese Patent Application No. 202211083012.6, filed with the China National Intellectual Property Administration on Sep. 6, 2022 and entitled “ABLATION SYSTEM”, which is incorporated herein by reference in its entirety.

### TECHNICAL FIELD

Embodiments of the present application relate to the technical field of medical instruments, in particular to an ablation system.

### BACKGROUND

The incidence rate of malignant tumors is increasing year by year, and the threat to human health is growing. Conventional treatment methods such as surgery, radiotherapy, and chemotherapy are increasingly mature, but inevitably incur varying degrees of damage to the normal functions of the body, and their therapeutic success rate still needs to be improved. With the development of science and technology, especially with the advancement of medical imaging technologies such as magnetic resonance imaging and ultrasonic imaging, minimally invasive ablation therapy for tumors, especially low-temperature cryoablation, has made a significant progress and is becoming increasingly popular.

Cryoablation devices currently on the market are mainly based on two principles. One is based on the gas throttling effect, namely, the Joule Thomson principle: high-pressure room temperature gases such as nitrogen and argon are cooled by throttling expansion to achieve cryoablation. For this type of cryoablation devices, infinite adjustment of gas flow is usually achieved by a proportional valve, thereby achieving continuous adjustment of cryogenic power. The other is to evaporate and absorb heat from human tissues through a cryogenic refrigerant such as liquid nitrogen. For this type of cryoablation devices that deliver cryogenic media such as liquid nitrogen, there is currently no proportional valve available on the market for use under extremely cryogenic conditions, so direct infinite adjustment for this type of cryogenic mode cannot be achieved.

The authorized patent High and Low Temperature Composite Ablation Surgical System (application number 201922147498.5) of HYGEA discloses a multi-channel refrigeration system based on the principle of cryogenic working medium refrigeration. In the system, a cold working medium discharged from a cold tank is divided into a gaseous working medium and a liquid working medium by a phase separator, the gaseous working medium is discharged out of the system, and the liquid working medium coming out of the phase separator is divided into four paths, which are connected to four solenoid valves and then delivered to four cryoablation needle channels respectively. Because the opening and closing of each channel need to be controlled separately, a cryogenic solenoid valve is directly arranged in each of the probe channels and the phase separator. That is, a large valve block is added between the phase separator and the probe. When the probe is used, the cold working medium cools the probe, the solenoid valve, and surrounding pipelines into the temperature of liquid nitrogen for operation. However, the probe is much smaller than the cryogenic solenoid valve. Under such a structure, the time from the opening of the system to the cooling of the probe to a minimum temperature is greatly prolonged, which reduces work efficiency.

In order to solve this problem, a pre-cooler is added on a solenoid valve base in the system. The gaseous working medium flowing out of the phase separator is first delivered to the pre-cooler and then discharged out of the system, such that the cooling capacity of the gaseous working medium is fully utilized, and the problem of excessively long cooling time is solved to some extent. Such improvement adds the pre-cooler, namely, further increases the volume of the valve block, so this problem is not solved essentially.

Therefore, a multi-channel ablation system with high work efficiency is urgently needed.

### SUMMARY

In view of the above problem, the present application provides an ablation system to overcome or at least partially solve the above problem.

An embodiment of the present application provides an ablation system including: a medium storage device used for storing a liquid medium; a plurality of probes, where each of the probes is provided with an air inlet channel and an air return channel; a liquid inlet module provided with a gas-liquid separation device, the gas-liquid separation device including a gas pipeline and a plurality of liquid pipelines, where each liquid pipeline can be communicated with or isolated from the air inlet channel of each probe; a heat exchange module provided with a heat exchanger, where the heat exchanger is communicated with the air return channel to convert the liquid medium delivered in the air return channel into a gas medium; and a control module electrically connected to each probe and the heat exchange module respectively, where the control module obtains the temperature of each probe and controls the flow rate Q2 of the gas medium flowing out of the heat exchange module.

Optionally, the ablation system further includes a first-type joint and a second-type joint, and the first-type joint can be clamped with or detached from the second-type joint; when the first-type joint is clamped with the second-type joint, each liquid pipeline is communicated with the air inlet channel of each probe; when the first-type joint is detached from the second-type joint, the first-type joint automatically closes to prevent the liquid medium in the liquid pipeline from flowing out.

Optionally, the first-type joint includes two first pipelines, the second-type joint includes two second pipelines, the two first pipelines are connected to the liquid pipeline and the heat exchanger respectively, and the two second pipelines are connected to the air inlet channel and the air return channel of the probe respectively; when the first-type joint is clamped with the second-type joint, the two first pipelines are communicated with the two second pipelines respectively.

Optionally, each first pipeline includes a first pipe segment, a second pipe segment, and a third pipe segment connected sequentially, where the inner diameter of the second pipe segment is greater than that of the first pipe segment and the third pipe segment, an elastic member and a ball are provided in an inner cavity of the second pipe segment, the elastic member presses against an end of the second pipe segment, and the inner diameter of the third pipe segment is less than the diameter of the ball and less than the inner diameter of the second pipe segment; when the first-type joint is detached from the second-type joint, the ball is clamped between the second pipe segment and the third pipe segment; the outer diameter of a first end of each second pipeline is less than the inner diameter of the third pipe segment, such that when the first-type joint is docked with the second-type joint, the first end of each second pipeline passes through the third pipe segment and enters the second pipe segment, and the ball moves away from an end of the third pipe segment.

Optionally, the second pipeline is provided with an extension portion and a first pore channel, where the extension portion extends radially outward along the first end of the second pipeline, and the first pore channel is formed between the extension portion and a body of the second pipeline.

Optionally, the heat exchange module further includes a first proportional valve, the first proportional valve is connected to the heat exchanger, and the control module controls the flow rate Q2 of the gas medium via the first proportional valve.

Optionally, the control module adjusts the flow rate Q2 of the gas medium according to the difference ΔT between the temperature T of the probe and a target temperature T2, to ensure that the temperature T of the probe tends towards the target temperature T2.

Optionally, the relationship between the flow rate Q2 of the gas medium and the target temperature T2 roughly conforms to an empirical formula: T2=a1-a2*a3\hat{ }Q2, where a1, a2, and a3 are constants.

Optionally, the heat exchanger is connected to a second proportional valve and the gas pipeline respectively, and the second proportional valve is used for controlling the flow rate of the gas medium from the gas pipeline.

Optionally, the liquid inlet module further includes a first solenoid valve, the first solenoid valve is connected to the gas-liquid separation device, and the control module is electrically connected to the first solenoid valve for controlling the liquid medium in the medium storage device to flow into the gas-liquid separation device.

Optionally, a liquid medium usage valve is provided between the first solenoid valve and the medium storage device.

Optionally, the ablation system further includes a reheating module, which includes a temperature control device and a second solenoid valve connected to the temperature control device, the temperature control device is communicated with the air inlet channel of the probe via a pipeline, and a gas medium usage valve is provided between the second solenoid valve and the medium storage device.

Optionally, the probe is further provided with a tube body, a needle, and a thermocouple wire; the air return channel surrounds the air inlet channel circumferentially, a vacuum layer is formed between the air return channel and the tube body, the thermocouple wire passes through the air return channel and enters at least a portion of the needle, and the thermocouple wire is electrically connected to the control module for transmitting the current temperature T of the probe to the control module.

Optionally, the gas-liquid separation device includes an inner casing and an outer casing, a channel is formed between the inner casing and the outer casing, a plurality of first through holes are spaced apart on a wall of the inner casing, an end of the outer casing is provided with a second through hole, and the second through hole is connected to the heat exchanger via a pipeline.

Optionally, an end of the gas-liquid separation device is provided with a plurality of second pore channels, each second pore channel is communicated with the inner casing, each second pore channel is also communicated with the air inlet channel of the probe, and the channel runs through the end of the gas-liquid separation device among the second pore channels.

From the above technical solution, it can be seen that the ablation system in the embodiments of the present application includes the plurality of probes and the gas-liquid separation device with the plurality of liquid pipelines, where the liquid pipeline of the gas-liquid separation device is directly communicated with the air inlet channel of the probe; after the medium enters the heat exchanger via the air return channel of the probe, each channel is controlled by the first proportional valve without adding any valve block between the gas-liquid separation device and the probe, thereby greatly shortening time for cooling the probe to a minimum temperature and improving work efficiency.

In the prior art, the flow rate of a liquid medium cannot be continuously controlled, so when cryoablation is performed on a patient's tumor, only an ice ball with a fixed size can be formed at the tumor. In the ablation system according to the embodiments of the present application, after the liquid medium flowing through the probe is completely gasified into room temperature gas, the flow rate of the liquid medium is continuously controlled by the first proportional valve, the cooling power of the medium is continuously adjusted, and the temperature of the probe is accurately controlled, such that ice balls with different sizes can be formed as required when cryoablation is performed on patients' tumors, and the tumors with different sizes can be consistent with a cryogenic range by adjusting the flow rate of the liquid medium or the temperature of the probe. On the other hand, for tumors at margins of some important organs, the cryoablation can be prevented from injuring the important organs by adjusting the flow rate of the liquid medium or the temperature of probe.

### BRIEF DESCRIPTION OF THE DRAWINGS

In order to describe the technical solutions in the embodiments of the present application or in the prior art more clearly, the accompanying drawings that need to be used in the description of the embodiments or the prior art will be briefly introduced below. Apparently, the accompanying drawings in the description below are merely some embodiments recited in the embodiments of the present application, and those of ordinary skill in the art may also derive other drawings according to these drawings without creative efforts.

FIG. 1 is a schematic view of an ablation system in one embodiment of the present application;

FIGS. 2A to 2B are cross-sectional views of a first-type joint and a second-type joint in one embodiment of the present application in an insertion state and a detachment state, respectively;

FIGS. 2C-2D are partially enlarged cross-sectional views of the first-type joint in the present application in the detachment state and the insertion state, respectively;

FIGS. 3A-3B are cross-sectional views of the first-type joint and the second-type joint in another embodiment of the present application in the insertion state and the detachment state, respectively;

FIGS. 3C-3D are cross-sectional views of the first-type joint and a plug in the present application in the detachment state and the insertion state, respectively;

FIG. 4A is a schematic view of pipeline connection of a gas-liquid separation device in the ablation system of the present application;

FIG. 4B is an enlarged schematic view of a portion including the gas-liquid separation device in the present application;

FIG. 4C is a front view of the gas-liquid separation device in one embodiment of the present application;

FIGS. 4D-4E are cross-sectional views taken along line A-A and line B-B in FIG. 4C, respectively;

FIG. 5 is a front perspective view of a heat exchange module in one embodiment of the present application;

FIG. 6 is a rear perspective view of the heat exchange module in one embodiment of the present application; and

FIG. 7 is a cross-sectional view of a probe in one embodiment of the present application.

### REFERENCE NUMERALS OF ELEMENTS

10: Ablation system; 101: Medium storage device; 102: Probe; 1021: Air inlet channel; 1022: Air return channel; 103: Liquid inlet module; 1031: Gas-liquid separation device; 1032: Gas pipeline; 1033: Liquid pipeline; 104: Heat exchange module; 1041: Heat exchanger; 105: Control module; 1042: Third interface; 1043: Fourth interface; 1034: First interface; 1035: Second interface; 1044: First proportional valve; 1045: Second proportional valve; 1036: First solenoid valve; 106: Reheating module; 1061: Temperature control device; 1062: Second solenoid valve; 1023: Tube body; 1024: Needle 1025: Thermocouple wire; 1026: Vacuum layer; 1037: Inner casing; 1038: Outer casing; 1039: Through hole; 1131: Pressure sensor interface; 1132: Thermocouple interface; 1046: Exhaust port; 1047: Fan; 1048, 1049: Flow meter; 21: First-type joint; 210, 210a, 210b: First pipeline; 2101, 2103: First end of the first pipeline; 2102, 2104: Second end of the first pipeline; 201: First pipe segment; 202: Second pipe segment; 203: Third pipe segment; 204: Elastic member; 205: Ball; 221: Elastic buckle; 206: Groove; 222: Baffle; 2011: First end of the second pipe segment; 2012: Second end of the second pipe segment; 220, 220a, 220b: Second pipeline; 223, 2230: First end of the second pipeline; 2231, 2232: Second end of the second pipeline; 224: Extension portion; 225: Body of the second pipeline; 226: First pore channel; 227: Sealing ring; 300: Plug; 401: Second pore channel; 1039: First through hole; 402: Second through hole; 403: End of the gas-liquid separation device; 404: Channel; 22: Second-type joint.

### DETAILED DESCRIPTION OF EMBODIMENTS

To make those skilled in the art better understand the technical solutions in the embodiments of the present application, the following clearly and completely describes the technical solutions in the embodiments of the present application with reference to the accompanying drawings therein. Apparently, the described embodiments are merely some but not all of the embodiments of this disclosure. All other embodiments obtained by those of ordinary skill in the art based on the embodiments of the present application should fall within the scope of protection of the embodiments of the present application.

The following further describes specific implementations of the embodiments of the present application with reference to the accompanying drawings.

With reference to FIG. 1 to FIG. 7, in a specific implementation of the present application, an ablation system 10 is provided, including: a medium storage device 101 used for storing a liquid medium; a plurality of probes 102 each provided with an air inlet channel 1021 and an air return channel 1022; a liquid inlet module 103 provided with a gas-liquid separation device 1031, the gas-liquid separation device 1031 including a gas pipeline 1032 and a plurality of liquid pipelines 1033, where each liquid pipeline 1033 can be communicated with or isolated from the air inlet channel 1021 of each probe 102; a heat exchange module 104 provided with a heat exchanger 1041, where the heat exchanger 1041 is communicated with the air return channel 1022 to convert the liquid medium delivered in the air return channel 1022 into a gas medium; and a control module 105 electrically connected to the probe 102 and the heat exchange module 104 respectively, where the control module 105 obtains the temperature of the probe 102 and controls the flow rate Q2 of the gas medium flowing out of the heat exchange module 104, to ensure that the temperature T of the probe 102 tends towards a target temperature T2.

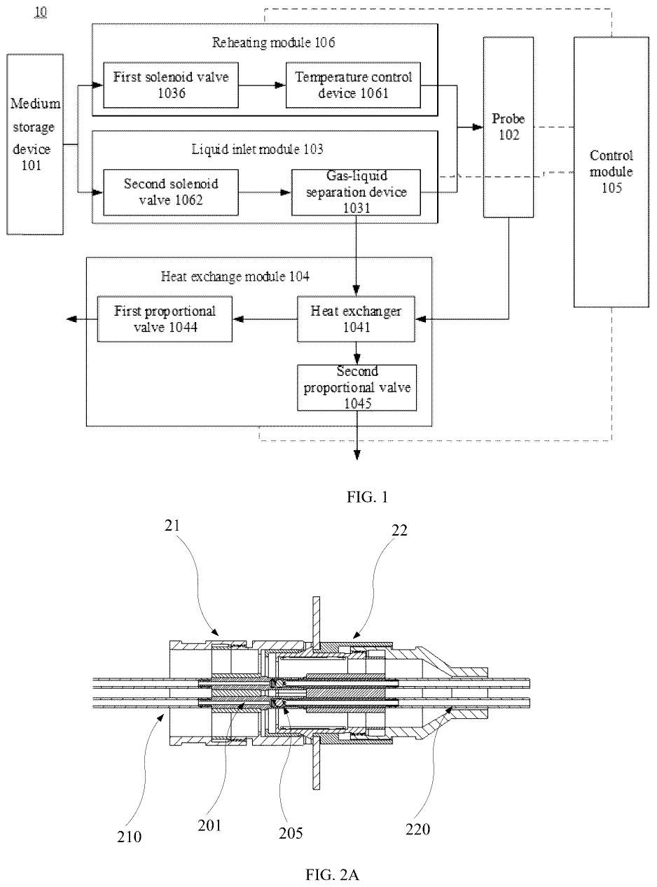

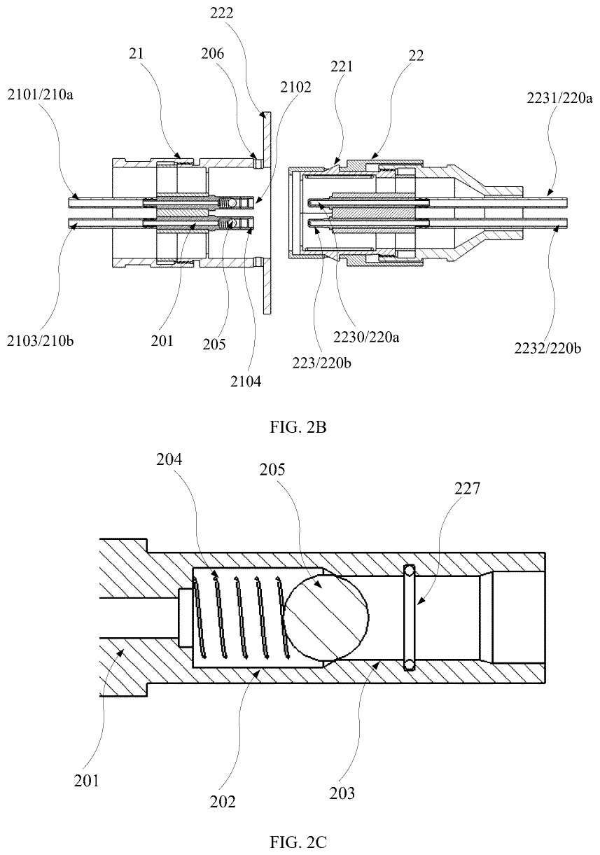

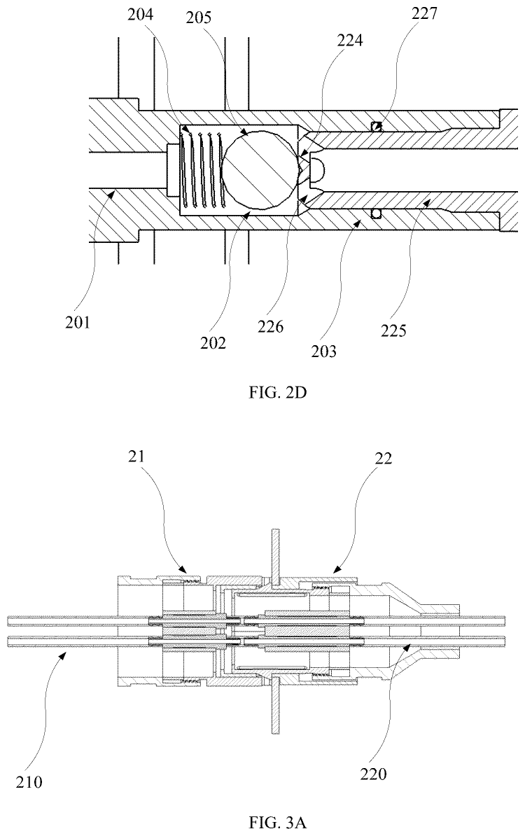

As shown in FIGS. 2A to 2D, in one embodiment, the ablation system 10 further includes a first-type joint 21 and a second-type joint 22, and the first-type joint 21 can be clamped with or detached from the second-type joint 22; when the first-type joint 21 is clamped with the second-type joint 22, each liquid pipeline is communicated with the air inlet channel of each probe; when the first-type joint 21 is detached from the second-type joint 22, the first-type joint 21 automatically closes to prevent the liquid medium in the liquid pipeline from flowing out.

The first-type joint 21 may be provided with a groove 206, and the second-type joint 22 may be provided with an elastic buckle 221. When the first-type joint 21 is clamped with the second-type joint 22, the elastic buckle 221 is compressed by downward pressure of a baffle 222 arranged at an end of the first-type joint 21, then the elastic buckle 221 enters the groove 206 and restores to its previous state, and the elastic buckle 221 is clamped with the groove 206. When the first-type joint 21 is detached from the second-type joint 22, pressure can be applied to the elastic buckle 221 via the groove 206, such that the elastic buckle 221 is compressed and ejected from the groove 206.

In an optional embodiment, the first-type joint 21 includes two first pipelines 210, the second-type joint 22 includes two second pipelines 220, a first end 2101 of the first pipeline 210a is communicated with the liquid pipeline of the gas-liquid separation device 1031, a first end 2103 of the first pipeline 210b is communicated with the heat exchanger, a second end 2102 of the first pipeline 210a and a second end 2104 of the first pipeline 210b are used for being communicated with a first end 223 of the second pipeline 220a and a first end 2230 of the second pipeline 220b, a second end 2231 of the second pipeline 220a is communicated with the air inlet channel 1021 of the probe 102, and a second end 2232 of the second pipeline 220b is communicated with the air return channel 1022 of the probe 102. When the first-type joint 21 is clamped with the second-type joint 22, the first pipeline 210a is communicated with the second pipeline 220a, the first pipeline 210b is communicated with the second pipeline 220b, and the liquid medium flows through the first pipeline 210a and the second pipeline 220a sequentially from the liquid pipeline of the gas-liquid separation device 1031 and enters the air inlet channel of the probe; the liquid medium flowing out of the air return channel of the probe flows through the second pipeline 220b and the first pipeline 210b sequentially and then enters the heat exchanger.

Each first pipeline (210a, 210b) includes a first pipe segment 201, a second pipe segment 202, and a third pipe segment 203 connected sequentially, where the inner diameter of the second pipe segment 202 is greater than that of the first pipe segment 201 and the third pipe segment 203, an elastic member 204 and a ball 205 are provided in an inner cavity of the second pipe segment 202, the elastic member 204 presses against a first end 2011 of the second pipe segment 202, the ball 205 is close to a second end 2012 of the second pipe segment 202, and the inner diameter of the third pipe segment 203 is less than the diameter of the ball 205 and less than the inner diameter of the second pipe segment 202. The elastic member 204 may be a spring. The outer diameter of the first end 223 of each second pipeline is less than the inner diameter of the third pipe segment 203, such that when the first-type joint 21 is clamped with the second-type joint 22, the first end (223, 2230) of each second pipeline passes through the third pipe segment 203 and enters the second pipe segment 202, the ball 205 clamped between the second pipe segment 202 and the third pipe segment 203 is squeezed by the first end (223, 2230) of the second pipeline to compress the elastic member 204, and the ball 205 moves away from the end of the third pipe segment 203.

The first end 223 of the second pipeline may further be provided with an extension portion 224 extending radially outward along the first end 223 of the second pipeline, and a first pore channel 226 formed between the extension portion 224 and a body 225 of the second pipeline. When the ball 205 presses against the first end of the second pipeline, the medium can enter the third pipe segment 203 from the second pipe segment 202 via the first pore channel 226. An inner wall of the third pipe segment 203 may be provided with a sealing ring 227, which is used for increasing the sealing property between the first end of the second pipeline and the first pipeline when the first end of the second pipeline is inserted, to prevent the medium from leaking out.

When the first-type joint 21 is detached from the second-type joint 22, the first end of the second pipeline gradually withdraws from the third pipe segment 203, and the ball 205 and the elastic member 204 move towards the third pipe segment 203 until the ball 205 is clamped between the second pipe segment 202 and the third pipe segment 203, such that the medium in the second pipe segment 202 cannot flow into the third pipe segment 203, and therefore cannot leak out from the first-type joint 21. As such, when a certain probe needs to be used, the first-type joint 21 and the second-type joint 22 of the probe are clamped with each other, and the medium enters the air inlet channel of the probe from the liquid pipeline via the first-type joint 21 and the second-type joint 22, returns from the air return channel of the probe, and then enters the heat exchanger for gasification via the first-type joint 21 and the second-type joint 22; When the probe is not needed, the first-type joint 21 is detached from the second-type joint 22, and the first-type joint 21 of the probe automatically closes.

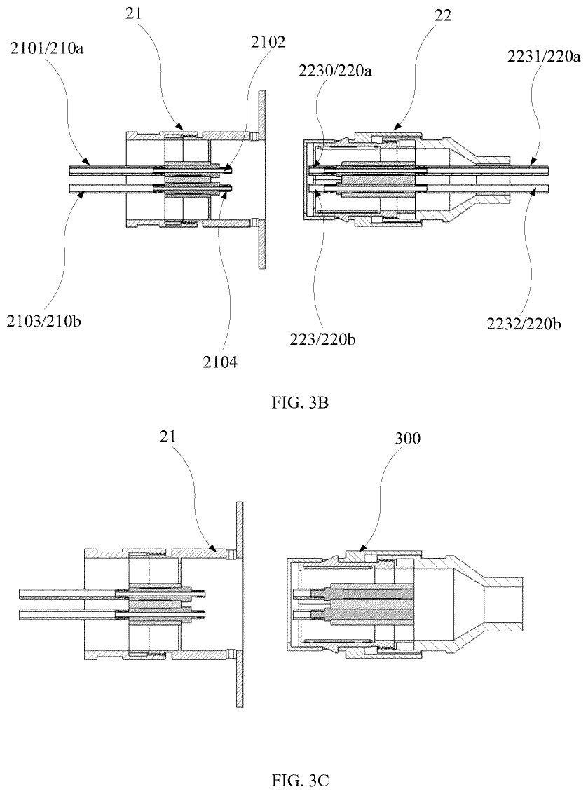

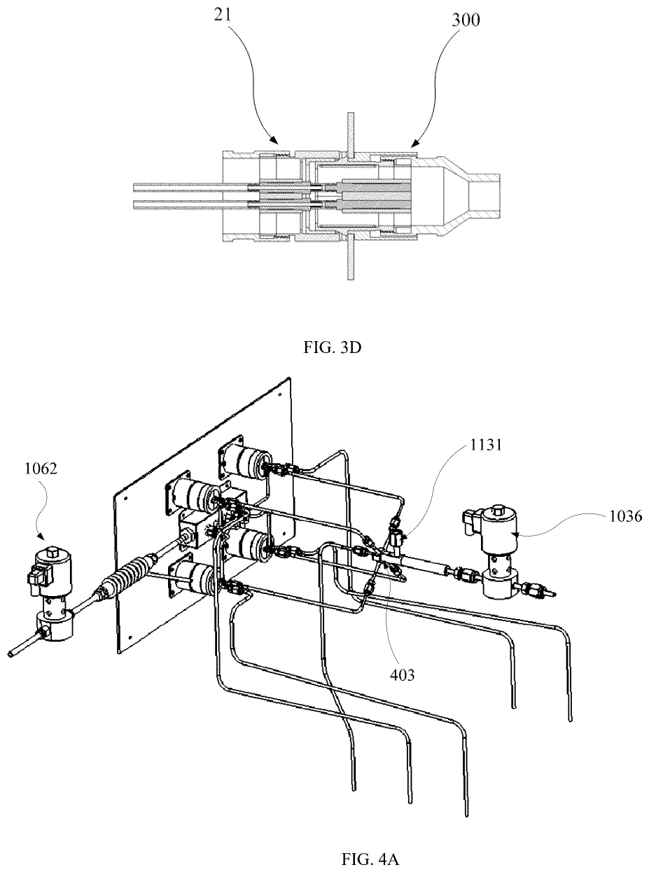

As shown in FIGS. 3A-3D, in another embodiment, the first-type joint 21 and the second-type joint 22 have a simple insertion relationship, and the first-type joint 21 does not have an automatic closing function. The outer diameters of the second ends (2102, 2104) of the two first pipelines (210a, 210b) may be less than the inner diameters of the first ends (223, 2230) of the second pipelines (220a, 220b). When the first-type joint 21 is inserted into the second-type joint, the second ends (2102, 2104) of the two first pipelines (210a, 210b) are inserted into the first ends (223, 2230) of the second pipelines (220a, 220b) to form a tight fit. In this embodiment, when a certain probe needs to be used, the first-type joint 21 and the second-type joint 22 of the probe are inserted; when the probe is not needed, the first-type joint 21 is detached from the second-type joint 22, and the first-type joint 21 is sealed with a plug 300. The end, docked with the first-type joint 21, of the plug 300 has two pipelines, while the other end has a closed structure that can prevent the medium from flowing out.

The medium may be nitrogen, argon, etc. The liquid medium is correspondingly liquid nitrogen, liquid argon, etc. The gas medium is correspondingly nitrogen, argon, etc. The medium storage device 101 may store a liquid cryogenic medium. The temperature of liquid nitrogen is related to pressure in an equilibrium state. For example, when the set working pressure is 1.0 Mpa, the corresponding temperature of liquid nitrogen is about −169 °C.

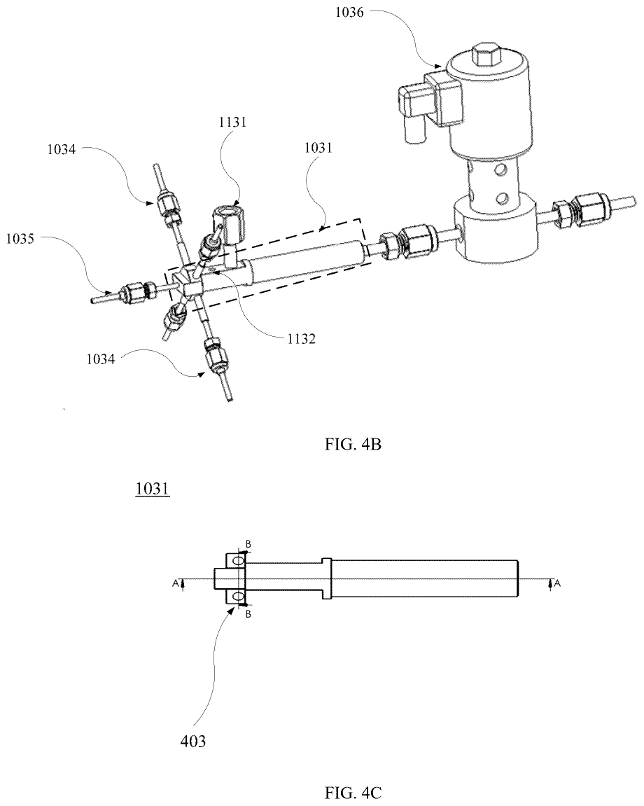

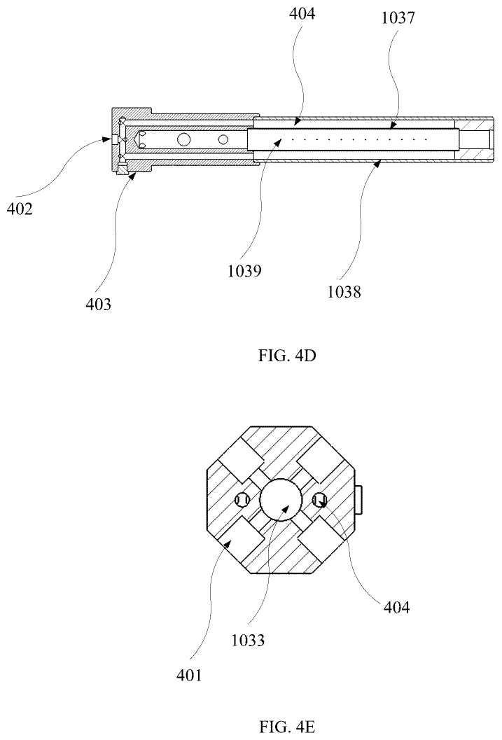

As shown in FIGS. 4A-4B, in one embodiment, the gas-liquid separator may be further provided with a pressure sensor interface 1131 and a thermocouple interface 1132, the pressure sensor interface 1131 is connected to a pressure sensor, and the pressure sensor is electrically connected to the control module 105 to monitor the pressure inside the ablation system 10 in real time; the thermocouple interface 1132 is connected to a thermocouple, and the thermocouple is electrically connected to the control module 105 to detect the temperature inside the ablation system 10. The gas-liquid separator may be further provided with a first interface 1034 and a second interface 1035, which are communicated with the air inlet channel 1021 of the probe 102 and the heat exchanger 1041 respectively.

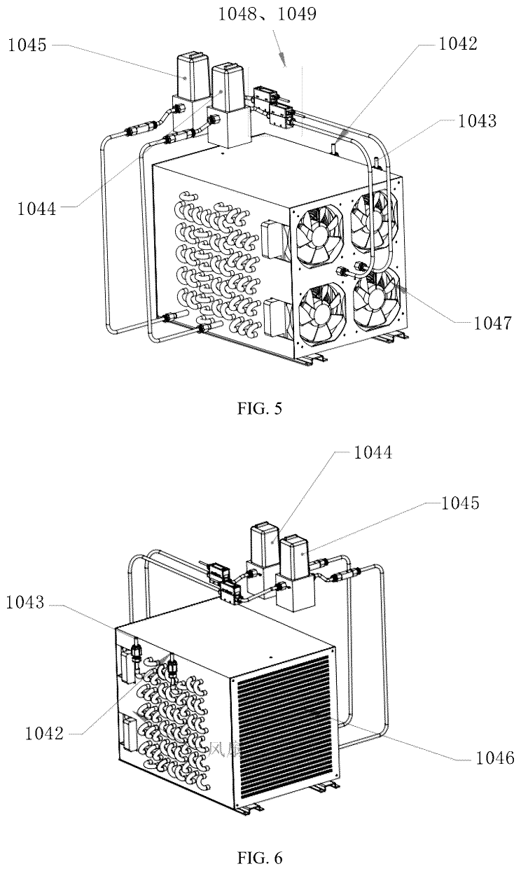

As shown in FIG. 5 and FIG. 6, in one embodiment, the heat exchange module 104 further includes a first proportional valve 1044, the first proportional valve 1044 is connected to the heat exchanger 1041, and the control module 105 controls the flow rate Q of the gas medium via the first proportional valve 1044. Because the volume ratio of nitrogen to liquid nitrogen is a fixed constant under constant pressure, the flow rate of liquid nitrogen can be continuously adjusted by adjusting the flow rate of nitrogen, that is, the flow rate of liquid nitrogen can be continuously adjusted by adjusting the flow rate Q of the gas medium via the first proportional valve 1044.

The heat exchanger 1041 in the heat exchange module 104 is a high-efficiency heat exchanger, which may be a finned heat exchanger, a plate heat exchanger, a shell and tube heat exchanger, etc. The heat exchanger 1041 may be provided with at least two independent heat exchange channels, which are communicated with the gas pipeline 1032 of the gas-liquid separation device 1031 and the air return channel 1022 of the probe 102 respectively. After sufficient heat exchange by the high-efficiency heat exchanger, the liquid nitrogen flowing out via the air return channel 1022 of the probe 102 is completely gasified into room temperature nitrogen (>−20 °C). Then, the room temperature nitrogen flows through the first proportional valve 1044 to control the flow rate Q2 of nitrogen. The first proportional valve 1044 may be a pressure proportional valve or a flow proportional valve.

In one embodiment, the control module 105 adjusts the flow rate Q2 of the gas medium according to the difference ΔT between the current temperature T of the probe 102 and the target temperature T2 (i.e. ΔT=T−T2), such that the temperature of the probe 102 tends towards the target temperature T2. In one embodiment, the relationship between the flow rate Q2 of the gas medium and the target temperature T2 roughly conforms to an empirical formula: T2=a1−a2*a3\hat{ }Q2, where a1, a2, and a3 are constants. The so-called empirical formula refers to a formula summarized based on experience. The relationship between variables in the formula does not fully conform to the definition in the formula, but is only an approximate expression. For example, the relationship between Q2 and T2 does not strictly follow the definition in the above formula.

An embodiment of the present application provides an adjustment process of the ablation system 10:

Firstly, the first proportional valve 1044 is fully opened or placed at a relatively high flow rate Q1 of nitrogen until the measured temperature of the probe 102 drops to a minimum temperature T1; during this process, the flow rate Q1 of nitrogen flowing through the first proportional valve 1044 gradually increases until stable. Then the first proportional valve 1044 is adjusted until the flow rate of nitrogen is Q2. According to the empirical formula T2=a1−a2*a3\hat{ }Q2, if Q2 is kept unchanged, the temperature T of the probe 102 will deviate from T2, and cannot be maintained at constant T2, that is, ΔT is not equal to 0 °C. Therefore, in order to maintain the temperature of the probe 102 at T2, that is, ΔT tends to 0 °C, when ΔT is not equal to 0 °C, Q2 needs to be controlled to ensure that the temperature T of the probe 102 always tends to T2.

In one embodiment, the heat exchanger 1041 is connected to a second proportional valve 1045 and the gas pipeline 1032 respectively, and the second proportional valve 1045 is used for controlling the flow rate of the gas medium from the gas pipeline 1032. After the pipelines of the system are fully pre-cooled, the flow rate of nitrogen discharged from the gas pipeline 1032 of the gas-liquid separator can be decreased by the second proportional valve 1045 to decrease the flow rate of liquid nitrogen, so as to reduce the waste of liquid nitrogen. Flow meters 1048 and 1049 may be provided on the first proportional valve 1044 and the second proportional valve 1045 to measure the flow rate of gas flowing through the first proportional valve 1044 and the second proportional valve 1045 respectively.

The heat exchanger 1041 may be provided with a third interface 1042 and a fourth interface 1043, which are communicated with the gas pipeline 1032 of the gas-liquid separation device 1031 and the air return channel 1022 of the probe 102 respectively. The heat exchanger 1041 may be further provided with an exhaust port 1046 and a fan 1047, which are used for discharging the gas medium and dissipating heat respectively.

In one embodiment, the liquid inlet module 103 further includes a first solenoid valve 1036, the first solenoid valve 1036 is connected to the gas-liquid separation device 1031, and the control module 105 is electrically connected to the first solenoid valve 1036 for controlling the liquid medium in the medium storage device 101 to flow into the gas-liquid separation device 1031. A liquid medium usage valve, such as a liquid nitrogen usage valve, may be provided between the medium storage device 101 and the first solenoid valve 1036 to control the discharge of the liquid medium in the medium storage device 101.

In one embodiment, the ablation system 10 further includes a reheating module 106, which includes a temperature control device 1061 and a second solenoid valve 1062 connected to the temperature control device 1061, the temperature control device 1061 is communicated with the air inlet channel 1021 of the probe 102 via a pipeline, and a gas medium usage valve is provided between the second solenoid valve 1062 and the medium storage device 101. After the probe 102 completes cryoablation, the second solenoid valve 1062 can be opened to deliver heated nitrogen, such that the probe 102 quickly restores to room temperature.

As shown in FIGS. 4A-4E, the gas-liquid separation device 1031 includes an inner casing 1037 and an outer casing 1038, a channel 404 is formed between the inner casing 1037 and the outer casing 1038, a plurality of first through holes 1039 are spaced apart on a wall of the inner casing 1037, an end of the outer casing 1038 is provided with a second through hole 402, and the second through hole 402 is connected to the heat exchanger 1041 via a pipeline. The inner casing 1037 is used for allowing the liquid medium to flow. After passing through the first through holes 1039, the liquid medium turns into gas, which flows in the outer casing 1038, then flows out of the second through hole 402, and flows into the heat exchanger 1041. An end 403 of the gas-liquid separation device 1031 is provided with a plurality of second pore channels 401, each second pore channel 401 is communicated with the inner casing 1037, and the channel 404 runs through the end 403 of the gas-liquid separation device 1031 among the second pore channels 401. The gas medium flows in the channel of the outer casing 1038, passes through the end 403 of the gas-liquid separation device 1031, concentrates and then flows out from the second through hole 402.

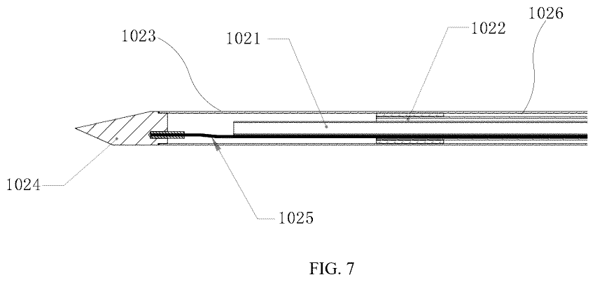

As shown in FIG. 7, in one embodiment, the probe 102 is further provided with a tube body 1023, a needle 1024, and a thermocouple wire 1025. The air return channel 1022 surrounds the air inlet channel 1021 circumferentially, and a vacuum layer 1026 is formed between the air return channel 1022 and the tube body 1023. The thermocouple wire 1025 passes through the air return channel 1022 and enters at least a portion of the needle 1024. The thermocouple wire 1025 entering the needle 1024 can be covered with a hard material such as stainless steel. The other end of the thermocouple wire 1025 is electrically connected to the control module for transmitting the current temperature T of the probe 102 to the control module.

From the above technical solution, it can be seen that the ablation system in the embodiments of the present application includes the plurality of probes and the gas-liquid separation device with the plurality of liquid pipelines, where the liquid pipeline of the gas-liquid separation device is directly communicated with the air inlet channel of the probe; after the medium enters the heat exchanger via the air return channel of the probe, each channel is controlled by the first proportional valve without adding any valve block between the gas-liquid separation device and the probe, thereby greatly shortening time for cooling the probe to a minimum temperature and improving work efficiency.

In the prior art, the flow rate of a liquid medium cannot be continuously controlled, so when cryoablation is performed on a patient's tumor, only an ice ball with a fixed size can be formed at the tumor. In the ablation system 10 according to the embodiments of the present application, after the liquid medium flowing through the probe 102 is completely gasified into room temperature gas, the flow rate of the liquid medium is continuously controlled by the first proportional valve 1044, the cooling power of the medium is continuously adjusted, and the temperature of the probe 102 is accurately controlled, such that ice balls with different sizes can be formed as required when cryoablation is performed on patients' tumors, and the tumors with different sizes can be consistent with a cryogenic range by adjusting the flow rate of the liquid medium or the temperature of the probe 102. On the other hand, for tumors at margins of some important organs, the cryoablation can be prevented from injuring the important organs by adjusting the flow rate of the liquid medium or the temperature of probe 102.

Finally, it should be noted that the above embodiments are merely used for explaining the technical solutions of the embodiments of the present application, rather than limiting the present application. Although the present application is described in detail with reference to the above embodiments, those of ordinary skill in the art should understand that they can still make modifications to the technical solutions described in the above embodiments or equivalent replacements to some technical features therein, without departing from the spirit and scope of the technical solutions of the embodiments of the present application.

1-10. (canceled)

11. An ablation system, wherein the ablation system comprises:
a medium storage device used for storing a liquid medium;
a plurality of probes, wherein each of the probes is provided with an air inlet channel and an air return channel;
a liquid inlet module provided with a gas-liquid separation device, the gas-liquid separation device comprising a gas pipeline and a plurality of liquid pipelines, wherein each liquid pipeline is enable to be communicated with or isolated from the air inlet channel of each probe;
a heat exchange module provided with a heat exchanger, wherein the heat exchanger is communicated with the air return channel to convert the liquid medium delivered in the air return channel into a gas medium; and
a control module electrically connected to each probe and the heat exchange module respectively, wherein the control module obtains the temperature of each probe and controls the flow rate Q2 of the gas medium flowing out of the heat exchange module.

12. The ablation system according to claim 1, wherein the ablation system further comprises a first-type joint and a second-type joint, and the first-type joint is enable to be clamped with or detached from the second-type joint; when the first-type joint is clamped with the second-type joint, each liquid pipeline is communicated with the air inlet channel of each probe; when the first-type joint is detached from the second-type joint, the first-type joint automatically closes to prevent the liquid medium in the liquid pipeline from flowing out.

13. The ablation system according to claim 2, wherein the first-type joint comprises two first pipelines, the second-type joint comprises two second pipelines, the two first pipelines are connected to the liquid pipeline and the heat exchanger respectively, and the two second pipelines are connected to the air inlet channel and the air return channel of the probe respectively; when the first-type joint is clamped with the second-type joint, the two first pipelines are communicated with the two second pipelines respectively.

14. The ablation system according to claim 3, wherein each of the first pipelines comprises a first pipe segment, a second pipe segment, and a third pipe segment connected sequentially, wherein the inner diameter of the second pipe segment is greater than that of the first pipe segment and the third pipe segment, an elastic member and a ball are provided in an inner cavity of the second pipe segment, the elastic member presses against an end of the second pipe segment, and the inner diameter of the third pipe segment is less than the diameter of the ball and less than the inner diameter of the second pipe segment; when the first-type joint is detached from the second-type joint, the ball is clamped between the second pipe segment and the third pipe segment; the outer diameter of a first end of each second pipeline is less than the inner diameter of the third pipe segment, such that when the first-type joint is docked with the second-type joint, the first end of each second pipeline passes through the third pipe segment and enters the second pipe segment, and the ball moves away from an end of the third pipe segment.

15. The ablation system according to claim 3, wherein the second pipeline is provided with an extension portion and a first pore channel, wherein the extension portion extends radially outward along the first end of the second pipeline, and the first pore channel is formed between the extension portion and a body of the second pipeline.

16. The ablation system according to claim 1, wherein the heat exchange module further comprises a first proportional valve, the first proportional valve is connected to the heat exchanger, and the control module controls the flow rate Q2 of the gas medium via the first proportional valve.

17. The ablation system according to claim 1, wherein the control module adjusts the flow rate Q2 of the gas medium according to the difference ΔT between a temperature T of the probe and a target temperature T2, to ensure that the temperature T of the probe tends towards the target temperature T2.

18. The ablation system according to claim 7, wherein the relationship between the flow rate Q2 of the gas medium and the target temperature T2 roughly conforms to an empirical formula: T2=a1−a2*a3\hat{ }Q2, wherein a1, a2, and a3 are constants.

19. The ablation system according to claim 5, wherein the gas-liquid separation device comprises an inner casing and an outer casing, a channel is formed between the inner casing and the outer casing, a plurality of first through holes are spaced apart on a wall of the inner casing, an end of the outer casing is provided with a second through hole, and the second through hole is connected to the heat exchanger via a pipeline.

20. The ablation system according to claim 9, wherein an end of the gas-liquid separation device is provided with a plurality of second pore channels, each second pore channel is communicated with the inner casing, each second pore channel is also communicated with the air inlet channel of the probe, and the channel runs through the end of the gas-liquid separation device among the second pore channels.
